(19) United States
(12) Patent Application Publication
(10) Pub. No.: US 2017/0341980 A1
(43) Pub. Date: Nov. 30, 2017

(54) NOBLE GAS NEUROPROTECTION AND NEUROREGENERATION FROM TREATMENT RELATED NEUROTOXICITY

(71) Applicant: NOBLIS THERAPEUTICS, INC., Portland, OR (US)

(72) Inventors: Vlad Bogin, Portland, OR (US); Thomas Ichim, San Diego, CA (US); Santos Kesari, Santa Monica, CA (US)

(21) Appl. No.: 15/610,362

(22) Filed: May 31, 2017

Related U.S. Application Data
(60) Provisional application No. 62/343,620, filed on May 31, 2016.

(51) Int. Cl. C04B 22/00; C01B 21/24; A61K 33/00

(52) U.S. Cl. CPC C04B 22/0053; C01B 21/24; A61K 33/00

(57) ABSTRACT

Disclosed are means of inducing neuroregeneration and/or neuroprotection in patients with damage to the nervous system. In one embodiment, Noble Gas containing compositions are administered to a patient suffering from a neurological injury, said therapy is administered alone, or in combination with other therapies useful in the induction of neuronal protection/stimulation of neurogenesis. In one specific embodiment, patients are treated with Noble Gas compositions to restore neural function subsequent to radiation therapy or chemotherapy for neoplasia of the brain. In another embodiment, Noble Gas compositions are administered prior to, concurrent with or after radiation and chemotherapy in order to induce a protective effect on non-malignant cells without substantially interfering with efficacy of radiation and chemotherapy.

### CROSS-REFERENCE TO RELATED APPLICATIONS

This application claims priority to U.S. Provisional Application No. 62/343,620, filed May 31, 2016, which is incorporated herein by reference in its entirety.

### FIELD OF THE INVENTION

The invention relates to the field of tissue injury and radioprotectants, more specifically, the invention relates to selective production of non-malignant tissue by administration of Noble Gas containing compositions for neuroprotection and/or neuroregeneration.

### BACKGROUND OF THE INVENTION

Although irradiation of the brain has some success in treatment of cranial malignancies, one of the drawbacks is post-irradiation damage to non-malignant tissues, which leads to a variety of complications and cognitive deficiencies. One example is leukoencephalopathy is a side effect that occurs more commonly after chemotherapy, but can also occur after radiation therapy to the brain. Leukoencephalopathy usually affects the white matter of the brain. The white matter contains nerve cells covered with myelin (a substance that acts like an insulator and speeds up nerve signals). With leukoencephalopathy, the myelin sheaths (called demyelination) in the treated area and surrounding tissue are destroyed. Leukoencephalopathy is a late effect of radiation therapy to the brain and can be permanent. Symptoms include: lethargy, ataxia, numbness and in some cases seizures and death. To date no effective treatments alter the course of post radiation cognitive decline. Accordingly, novel means of preventing onset, and stimulation of regeneration are needed.

### SUMMARY OF THE INVENTION

Various aspects of the invention relating to the above are enumerated in the following paragraphs:

Aspect 1. A method of protecting non-neoplastic cells from cellular damaging effects of a brain cancer directed therapy, said method comprising the steps of: a) obtaining a Noble Gas composition with neuro protective and/or regenerative potential; and b) administering said composition in a manner to promote regeneration and/to provide selective protection of non-malignant tissue from effects of chemotherapy and/or radiation therapy.

Aspect 2. The method of aspect 1, wherein said brain cancer directed therapy comprises therapies selected from a group comprising of: a) radiation therapy; b) chemotherapy; c) surgery; d) metabolic therapy; e) targeted therapy, and f) immunotherapy.

Aspect 3. The method of aspect 1, wherein said Noble Gas composition is gas mixture containing oxygen and a proportion by volume of 20 to 70% of xenon.

Aspect 4. The method of aspect 3, wherein said proportion of xenon is between 22 and 60% by volume to oxygen.

Aspect 5. The method of aspect 4, wherein said proportion of xenon is between 25 and 60% by volume to oxygen.

Aspect 6. The method of aspect 1, wherein said noble gas containing mixture consists only of a) oxygen and xenon or b) air and xenon.

Aspect 7. The method of aspect 1, wherein said noble gas containing mixture also contains nitrogen, helium, Nitric Oxide, krypton, argon or neon.

Aspect 8. The method of aspect 1, wherein said noble gas containing mixture contains a proportion by volume of oxygen of between 15 and 25%.

Aspect 9. The method of aspect 1, wherein said noble gas containing mixture is supplied for inhalation from a pressurized container at a pressure greater than 2 bar.

Aspect 10. The method of aspect 1, wherein said noble gas containing mixture is administered intranasally.

Aspect 11. The method of aspect 1, wherein said noble gas containing mixture is administered through the use of a hyperbaric chamber.

Aspect 12. The method of aspect 11, wherein said hyperbaric chamber is pressurized to a pressure of no more than 3 atm (0.3 MPa).

Aspect 13. The method of aspect 12, wherein a noble gas is administered to the patient while the patient is in the hyperbaric environment.

Aspect 14. The method of aspect 1 wherein said noble gas is administered by inhalation or simulated inhalation.

Aspect 15. The method of aspect 1, wherein said noble gas is xenon, helium, or a mixture of xenon and helium.

Aspect 16. The method of aspect 1, wherein the noble gas is xenon or a mixture of xenon and helium, and the partial pressure of xenon is no more than about 0.8 atm (0.08 MPa).

Aspect 17. The method of aspect 1, wherein said noble gas is administered mixed with air, the air partial pressure being about 1 atm (0.1 MPa).

Aspect 18. The method of aspect 1, wherein said noble gas is administered as part of a gas mixture comprising oxygen, the nitrogen partial pressure in the mixture being equal to or less than about 0.8 atm (0.08 MPa).

Aspect 19. The method of aspect 18, wherein said gas mixture is essentially free of nitrogen.

Aspect 20. The method of aspect 19, wherein the oxygen partial pressure is about 0.2 atm (0.02 MPa).

Aspect 21. A method of preventing radiation injury during radiological treatment of a brain neoplasia comprising the steps of: a) identifying a patient prior to, concurrent with or after receiving radiation therapy and chemotherapy; b) administering to said patient a gaseous composition containing 25% xenon by volume; and c) administering said mixture for a sufficient time point and concentration to selectively upregulate bcl-2 in non-malignant cells, without substantially upregulating said bcl-2 in malignant cells.

Aspect 22. A method of preventing radiation injury during radiological treatment of a brain neoplasia comprising the steps of: a) identifying a patient prior to, concurrent with or after receiving radiation therapy and chemotherapy; b) administering to said patient a gaseous composition containing 25% xenon by volume; and c) administering said mixture for a sufficient time point and concentration to selectively upregulate p53 in malignant cells, without substantially upregulating said p53 in non-malignant cells.

Aspect 23. A method of selectively enhancing regeneration of non-malignant cells after a radiation and chemotherapy insult comprising the steps of: a) obtaining a Noble Gas composition with regenerative potential; and b) administering said composition in a manner to promote regeneration of non-malignant tissue from effects of chemotherapy and/or radiation therapy, while not enhancing growth or metastasis.

Aspect 24. The method of aspect 23, wherein said brain cancer directed therapy comprises therapies selected from a group comprising of: a) radiation therapy; b) chemotherapy; c) surgery; d) metabolic therapy; e) targeted therapy, and f) immunotherapy.

Aspect 25. The method of aspect 23, wherein said Noble Gas composition is gas mixture containing oxygen and a proportion by volume of 20 to 70% of xenon.

Aspect 26. The method of aspect 25, wherein said proportion of xenon is between 22 and 60% by volume to oxygen.

Aspect 27. The method of aspect 26, wherein said proportion of xenon is between 25 and 60% by volume to oxygen.

Aspect 28. The method of aspect 23, wherein said noble gas containing mixture consists only of a) oxygen and xenon or b) air and xenon.

Aspect 29. The method of aspect 23, wherein said noble gas containing mixture also contains nitrogen, helium, Nitric Oxide, krypton, argon or neon.

Aspect 30. The method of aspect 23, wherein said noble gas containing mixture contains a proportion by volume of oxygen of between 15 and 25%.

Aspect 31. The method of aspect 23, wherein said noble gas containing mixture is supplied for inhalation from a pressurized container at a pressure greater than 2 bar.

Aspect 32. The method of aspect 23, wherein said noble gas containing mixture is administered intranasally.

Aspect 33. The method of aspect 23, wherein said noble gas containing mixture is administered through the use of a hyperbaric chamber.

Aspect 34. The method of aspect 33, wherein said hyperbaric chamber is pressurized to a pressure of no more than 3 atm (0.3 MPa).

Aspect 35. The method of aspect 34, wherein a noble gas is administered to the patient while the patient is in the hyperbaric environment.

Aspect 36. The method of aspect 23 wherein said noble gas is administered by inhalation or simulated inhalation.

Aspect 37. The method of aspect 23, wherein said noble gas is xenon, helium, or a mixture of xenon and helium.

Aspect 38. The method of aspect 23, wherein the noble gas is xenon or a mixture of xenon and helium, and the partial pressure of xenon is no more than about 0.8 atm (0.08 MPa).

Aspect 39. The method of aspect 23, wherein said noble gas is administered mixed with air, the air partial pressure being about 1 atm (0.1 MPa).

Aspect 40. The method of aspect 23, wherein said noble gas is administered as part of a gas mixture comprising oxygen, the nitrogen partial pressure in the mixture being equal to or less than about 0.8 atm (0.08 MPa).

Aspect 41. The method of aspect 40, wherein said gas mixture is essentially free of nitrogen.

Aspect 42. The method of aspect 41, wherein the oxygen partial pressure is about 0.2 atm (0.02 MPa).

### DESCRIPTION OF THE INVENTION

The invention provides means of selectively protect healthy tissue from radiation or chemotherapy by administration of Noble Gases and compositions containing Noble Gases. In one embodiment the invention provides for administration of Noble Gases and/or compositions containing Noble Gases that selectively protect non-malignant brain tissue from irradiation or chemotherapy.

The invention provides compositions of matter, protocols and uses of Noble Gases aimed at reducing and/or ameliorating neurodegenerative effects of brain cancer targeted therapeutics. In one embodiment the invention teaches the use of Noble Gases with ability to provide anti-inflammatory and/or neuroprotective activities which mediate selective effects on non-malignant tissue, while allowing for chemotherapy and/or radiotherapy to target malignant tissue. In one specific embodiment, concentrations of Noble Gases are administered in a manner to selectively upregulate super oxide dismutase to non-neoplastic tissue, thus protecting endogenous non-malignant stem cells, for example in the dentate gyms and subventricular zone of the brain, while allowing for death, mitotic inactivation and autophagy of neoplastic cells found in the brain in response to radiation and/or chemotherapy. In another embodiment, Noble Gases are utilized post chemotherapy and/or radiation therapy to allow for amelioration of neurocognitive effects of said chemotherapy and/or radiation therapy.

It is known in the art that tumor cells lose specific physiological functions that are found in non-malignant cells in order to focus energy expenditure and cellular activities on proliferation, apoptosis resistance, and metastasis. Examples of such “focusing of resources” can be seen in the case of proteasomes, in which tumors lose several proteasomes found in non-malignant cells, thus reducing redundancy of protein degradation activity. Given activity, proteasome inhibitors such as bortezomib, have been shown to selectively kill cancer cells, which have lost redundancy, whereas healthy cells do not succumb to proteasome inhibition due to existing redundancy of protein degradation pathways [1]. Similarly, the current invention is based on the unexpected finding that tumor cells possess a reduced ability to evoke stem cell chemotactic responses after injury as compared to non-malignant brain tissue. In one embodiment the invention teaches the use of various stem cells for protection, treatment, and restoration of neurological function subsequent to chemotherapy and/or radiation therapy of brain tumors.

In one embodiment, the use of Noble Gas compositions is tailored to reduce upregulation of apoptosis in cell types that are targeted by radiation therapy. Specifically, it is known that radiation upregulates apoptosis in various cells of the central nervous system, including to regenerative neuronal populations.

One such radiosensitive cell type that is described by the inventors as a target of intervention are the cells in the dentate gyms. It is known that localized exposure of young rat brain to X-rays produces neuronal hypoplasia specific to the granule cell layer of the hippocampal dentate gyrus, which is believed to be responsible for endogenous neuroregenerative neuronal activity. This brain damage causes locomotor hyperactivity, slowed acquisition of passive avoidance tasks and long bouts of spontaneous turning [2]. Radiation induced neural deficit may be associated with altered distribution of afferent fibers in the molecular layer [3], reduction in NGF and BDNF [4], apoptosis of cells in the subgranular zone and the hilus of the dentate gyms [5]. In some embodiments of the invention, use of Noble Gas containing compositions are utilized as a means of upregulating expression of growth factors that are lost during events associated with administration of therapeutic radiation for the treatment of brain tumors.

The effects of radiation on healthy brain cells are often associated with induction of apoptosis. For example, Nagai et al systemically treated 90 four week old mice with 18 Gy X-rays (0.45 Gy/min); 10 each were decapitated and the cerebrums were removed 1, 3, 6, 9, 12, 18, 24, 48, and 72 hours after irradiation. Controls were 10 unirradiated mice. DNA fragmentation analysis was carried out by agarose gel electrophoresis, and morphological analysis was by the TUNEL method. Apoptosis of neuronal cells was detected by cerebral DNA ladders, which were visible between 6 to 24 hr, peaking in 9 hr. According to the TUNEL analysis, radiation-induced apoptosis increased, with a peak at 9 hours, but decreased 24 hours after irradiation. Apoptotic cells were always localized exclusively in the hippocampal dentate granule cells [6]. Apoptosis in brain cells has been observed in other systems in response to irradiation, and has been attributed, in part, to p53 activation [7]. One of the potent examples of the importance of p53 in upregulating apoptosis is the proclivity of cells lacking p53 to possess an inherent degree of radioresistance, as compared to cells expressing p53, which are relatively radiosensitive. Specifically in malignant brain tumors: medulloblastoma and glioblastoma, there is a profoundly different radiation responsiveness. Medulloblastoma is very sensitive to radiation therapy, whereas glioblastoma is highly resistant, and the long-term survival of medulloblastoma patients exceeds 50%, while there are few long-term survivors among glioblastoma patients. p53-mediated apoptosis is thought to be an important mechanism mediating the cytotoxic response of tumors to radiotherapy. In an experimental study, researchers compared the response to radiation of five cell lines that have wild-type p53: three derived from glioblastoma and two derived from medulloblastoma. They found that the medulloblastoma-derived cell lines underwent extensive radiation-induced apoptotic cell death, while those from glioblastomas did not exhibit significant radiation-induced apoptosis. p53-mediated induction of p21(BAX) is thought to be a key component of the pathway mediating apoptosis after the exposure of cells to cytotoxins, and the expression of mRNA encoding p21(BAX) was correlated with these cell lines undergoing radiation-induced apoptosis. The failure of p53 to induce p21(BAX) expression in glioblastoma-derived cell lines is likely to be of biologic significance, since inhibition of p21(BAX) induction in medulloblastoma resulted in a loss of radiation-induced apoptosis, while forced expression of p21(BAX) in glioblastoma was sufficient to induce apoptosis. The failure of p53 to induce p21(BAX) in glioblastoma-derived cell lines suggests a distinct mechanism of radioresistance [8].

Thus, in one embodiment of the invention, therapeutic Noble Gas compositions are administered in a manner to alter apoptotic molecules and their ratio in the body. Specifically, the invention teaches that various concentrations of xenon gas, when delivered into circulation, either by inhalation [9-11], or administration of echogenic xenon liposomes [12, 13], can be utilized to block p53 upregulation, and/or to block the ability of p53 to induce p21 (BAX). The use of xenon has been reviewed by numerous authors in the art, which provide guidance as to details of administration [14-16]. Importantly, the new and non-obvious aspect of the current invention is that application of neuroprotective properties of xenon to selective neuroprotection of non-malignant tissue, while allowing for radiation therapy to kill the tumor cells. It is to be noted that the invention envisions not only neuroprotection from radiation therapy, but from other therapies that are associated with efficacy in brain tumor treated, including in chemotherapy, targeted therapy, immunotherapy, and metabolic therapy.

In one embodiment, Noble Gas compositions are utilized that suppress p53 upregulation in non-malignant cells, while allowing for upregulation in neoplastic cells. The importance of p53 and means of analyzing its expression may be gleaned from previous publications in the art. For example, a study compared adult p53(+/+) to p53(−/−) mice exposed to gamma-irradiation. Apoptosis and neurogenesis were assessed up to 14 days following the injury. Five-ten hours following gamma-irradiation, cells with TUNEL positive nuclei were identified within the subgranular zone of dentate gyms (DG) of both p53(+/+) and p53(−/−) mice. At the same time-points, pyknotic and shrinking nuclei were visualized by Hoechst 33258 staining. Furthermore, gamma-irradiation increased the number of proliferating cell nuclear antigen (PCNA) positive cells with a peak at 5-10 h in both animal groups. PCNA immunoreactivity was detected in cells exhibiting condensed nuclei as visualized by Hoechst 33258 staining. Neurogenesis, assessed by mitotic marker p34(cdc2) immunoreactivity, showed a biphasic response to gamma-irradiation both in p53(+/+) and p53(−/−) mice which was characterized by an early inhibition and a delayed stimulation. In p53(−/−) mice, the time required by DG granule cells to recover from the lesion and to stimulate proliferation was significantly shortened in comparison with wild-type mice thus resulting in an accelerated neurogenesis. These data support the notion that following gamma-radiation p53 plays a role in regulating cell-cycle progression rate but it is dispensable for promoting apoptosis of DG granule cells [17].

Examples of gases or gas mixtures employed as medicament for radiation protection: 1.) 100% by volume xenon; 2.) 70% by volume xenon/30% by volume oxygen; 3.) 65% by volume xenon/30% by volume oxygen/5% by volume nitrogen; 4.) 65% by volume xenon/35% by volume oxygen; 5.) 60% by volume xenon/30% by volume oxygen/10% by volume nitrogen; 6.) 60% by volume xenon/35% by volume oxygen/5% by volume nitrogen; 7.) 60% by volume xenon/40% by volume oxygen; 8.) 55% by volume xenon/25% by volume oxygen/20% by volume nitrogen; 9.) 55% by volume xenon/30% by volume oxygen/15% by volume nitrogen; 10.) 55% by volume xenon/35% by volume oxygen/10% by volume nitrogen; 11.) 55% by volume xenon/40% by volume oxygen/5% by volume nitrogen; 12.) 55% by volume xenon/45% by volume oxygen; 13.) 50% by volume xenon/50% by volume oxygen; 14.) 50% by volume xenon/45% by volume oxygen/5% by volume nitrogen; 15.) 50% by volume xenon/40% by volume oxygen/10% by volume nitrogen; 16.) 50% by volume xenon/30% by volume oxygen/20% by volume nitrogen; 17.) 50% by volume xenon/25% by volume oxygen/25% by volume nitrogen; 18.) 45% by volume xenon/55% by volume oxygen; 19.) 45% by volume xenon/50% by volume oxygen/5% by volume nitrogen; 20.) 45% by volume xenon/45% by volume oxygen/10% by volume nitrogen; 21.) 45% by volume xenon/40% by volume oxygen/15% by volume nitrogen; 22.) 45% by volume xenon/35% by volume oxygen/20% by volume nitrogen; 23.) 45% by volume xenon/30% by volume oxygen/25% by volume nitrogen; 24.) 45% by volume xenon/30% by volume oxygen/25% by volume nitrogen; 25.) 40% by volume xenon/30% by volume oxygen/30% by volume nitrogen; 26.) 40% by volume xenon/50% by volume oxygen/10% by volume nitrogen; 27.) 35% by volume xenon/25% by volume oxygen/40% by volume nitrogen; 28.) 35% by volume xenon/65% by volume oxygen; 29.) 30% by volume xenon/70% by volume oxygen; 30.) 30% by volume xenon/50% by volume oxygen/20% by volume nitrogen; 31.) 30% by volume xenon/30% by volume oxygen/40% by volume nitrogen; 32.) 20% by volume xenon/80% by volume oxygen; 33.) 20% by volume xenon/30% by volume oxygen/50% by volume nitrogen; 34.) 15% by volume xenon/30% by volume oxygen/55% by volume nitrogen; 35.) 15% by volume xenon/50% by volume oxygen/35% by volume nitrogen; 36.) 10% by volume xenon/90% by volume oxygen; 37.) 10% by volume xenon/50% by volume oxygen/40% by volume nitrogen; 38.) 10% by volume xenon/30% by volume oxygen/60% by volume nitrogen; 39.) 10% by volume xenon/25% by volume oxygen/65% by volume nitrogen; 40.) 5% by volume xenon/25% by volume oxygen/70% by volume nitrogen; 41.) 5% by volume xenon/30% by volume oxygen/65% by volume nitrogen; 42.) 5% by volume xenon/50% by volume oxygen/45% by volume nitrogen; 43.) 5% by volume xenon/30% by volume oxygen/65% by volume nitrogen; 44.) 5% by volume xenon/95% by volume oxygen; 45.) 1% by volume xenon/99% by volume oxygen; 46.) 1% by volume xenon/30% by volume oxygen/69% by volume nitrogen; 47.) 1% by volume xenon/25% by volume oxygen/74% by volume nitrogen.

Xenon or a xenon-containing gas mixture are further used to produce a medicament for the treatment of impairments of blood flow in the brain, to produce a medicament for the treatment of impairment of cerebral perfusion, to produce a medicament for the treatment of cognitive impairments, to produce a medicament for cerebral protection specific to non-malignant cells, to produce a medicament for the prophylaxis and/or therapy of impairments of cognitive performance, also postirradiation, to produce a medicament for the treatment of irradiation associated fibrosis, to produce a medicament for the prophylaxis of white matter damage, to produce a medicament for improving the oxygen supply in the brain, to produce a medicament for the treatment of post-radiation associated ischemia syndrome, to produce a medicament for promoting blood flow in the brain as well as reduction in associated pathologies.

In addition, xenon or xenon-containing gas mixtures are advantageously employed as medicament for the treatment of states with oxygen deficiency associated with radiation damage, especially oxygen deficiency in the brain. For example, xenon or xenon-containing gas mixtures are employed in emergency situations such as the nuclear or dirty bombs. Xenon or a xenon-containing gas mixture is also used to produce a medicament for improving the oxygenation of the brain. Xenon or a xenon-containing gas mixture are further used to produce a medicament for the treatment of cognitive or cerebral dysfunction, in particular of postoperative cognitive dysfunction after neuronal irradiation. Cerebral dysfunctions caused by irradiation relate to impairments of the microcirculation, of oxygen utilization and of metabolic functions. The medicament is thus also used to treat cerebral disorders such as impairments of the microcirculation, of oxygen utilization and of metabolic functions.

Gaseous xenon or xenon-containing gas mixtures are particularly advantageously employed for prophylaxis before exposure to radiation therapy. Prophylactic administration of xenon or xenon-containing gas mixtures takes place for example preoperatively, intraoperatively or postoperatively.

The provided medicament for cerebral protection of nonmalignant tissue and the indications mentioned, or the medicament produced directly on use, in particular in the direct vicinity of the patient, is for example a gas mixture which comprises from 1 to 80% by volume (based on standard conditions, i.e. 20.degree. C., 1 bar absolute) xenon (e.g. remainder oxygen). The medicament which is administered to the patient comprises xenon in pharmacologically or therapeutically effective amount, in particular in subanesthetically or anesthetically effective amount. A medicament with xenon in subanesthetically effective amount is advantageous. Subanesthetically effective (subanesthetic) amounts of xenon mean those amounts or concentrations of xenon which are insufficient for general anesthesia. These are in general amounts of up to 70% by volume xenon, preferably up to 65% by volume, particularly preferably up to 60% by volume, in particular up to 50% by volume xenon. Pure xenon is accordingly metered into the patient's respiratory gas in the stated concentrations. This means that the respiratory gas supplied to the patient comprises for example from 5 to 60% by volume, 5 to 50% by volume, 5 to 40% by volume, 5 to 30% by volume or 5 to 20% by volume xenon. In special cases, e.g. for prophylaxis, especially during prolonged ventilation, a dosage of xenon in the respiratory gas with a low concentration, for example 1 to 35% by volume, 5 to 25% by volume or 5 to 20% by volume xenon in the respiratory gas, may be advantageous. The medicaments, in particular gaseous medicaments, preferably comprise besides xenon one or more gases or substances which are gaseous at body temperature under atmospheric pressure. Examples of gas mixtures which can be used are xenon-oxygen gas mixtures or gas mixtures of xenon and one or more inert gases such as nitrogen or a rare gas or xenon-oxygen inert gas mixtures. Admixture of a gas to the xenon may be very advantageous if it is intended to introduce little xenon into the body.

In one embodiment, Noble Gas containing mixtures are utilized to augment endogenous neural stem cells (NSCs) activity so as to preserve neural function against side effects of radiotherapy. NSC are self-renewing, multipotent stem cells that generate neurons, astrocytes and oligodendrocytes. The medical potential of neural stem cells is well-documented. Damaged central nervous system (CNS) tissue has very limited regenerative capacity so that loss of neurological function is often chronic and progressive. Neural stem cells (NSCs) have shown promising results in stem cell-based therapy of neurological injury or disease. Implanting neural stem cells (NSCs) into the brains of post-stroke animals has been shown to be followed by significant recovery in motor and cognitive tests. It is not completely understood how NSCs are able to restore function in damaged tissues but it is now becoming increasingly recognized that NSCs have multimodal repairing properties, including site-appropriate cell differentiation, pro-angiogenic and neurotrophic activity and immunomodulation promoting tissue repair by the native immune system and other host cells. It is likely that many of these effects are dependent on transient signaling from implanted neural stem cells to the host milieu, for example NSCs transiently express proinflammatory markers when implanted in ischemic muscle tissue damage which directs and amplifies the natural pro-angiogenic and regulatory immune response to promote healing and repair. In chronic stroke brain, NSCs also have a substantial neurotrophic effect. For example, they promote the repopulation of the stoke-damaged striatal brain tissue with host brain derived doublecortin positive neuroblasts.

### EXAMPLE

A clinical trial is conducted in 20 patients with brain metastasis who receive 2 Gy to 100 Gy fractionated over 2-8 weeks. 10 patients are treated with 10-20 liters of 30% xenon every other day during radiation exposure. An additional 10 patients are treated with similar radiation regimen and administered 10-20 liters of air every other day during radiation exposure. At 3, 8, 12, and 24 weeks, preservation in cognitive function as measured by HVLT-R Total Recall score (verbal learning and memory test) is observed in the xenon treated patients. Furthermore, verbal memory, as measured by the Hopkins Verbal Learning Test-Revised (HVLT-R), Cognitive flexibility as measured with the Controlled Oral Word Association (COWA), Word Fluency has measured with the Controlled Oral Word Association (COWA), Working memory has measured with the Wechsler Adult Intelligence Scale—Digit Span, Processing speed has measured with the Wechsler Adult Intelligence Scale—Digit Symbol, Motor dexterity has measured with the Grooved Pegboard (GP), Functional assessment, as measured by the Functional Assessment of Cancer Therapy-Brain (FACT-Br), all improved in the xenon treated group. No acceleration of tumor growth is seen in patients receiving xenon as compared to air controls.

### REFERENCES

1. Obeng, E. A., et al., Proteasome inhibitors induce a terminal unfolded protein response in multiple myeloma cells. Blood, 2006. 107(12): p. 4907-16.
2. Mickley, G. A., et al., Progressive behavioral changes during the maturation of rats with early radiation-induced hypoplasia of fascia dentata granule cells. Neurotoxicol Teratol, 1989. 11(4): p. 385-93.
3. Legendre, D. I., B. P. Vietje, and J. Wells, Traumatic lesions and transplants of granule cells in the dentate gyrus alter the distribution of afferent fibers in the molecular layer. J Neurotrauma, 1994. 11(3): p. 333-44.
4. Dimberg, Y., et al., Effects of X-irradiation on nerve growth factor in the developing mouse brain. Toxicol Lett, 1997. 90(1): p. 35-43.
5. Sasaki, R., et al., Target cells of apoptosis in the adult murine dentate gyrus and O4 immunoreactivity after ionizing radiation. Neurosci Lett, 2000. 279(1): p. 57-60.
6. Nagai, R., et al., Selective vulnerability to radiation in the hippocampal dentate granule cells. Surg Neurol, 2000. 53(5): p. 503-6; discussion 506-7.
7. Lang, F. F., et al., Enhancement of radiosensitivity of wild-type p53 human glioma cells by adenovirus-mediated delivery of the p53 gene. J Neurosurg, 1998. 89(1): p. 125-32.
8. Shu, H. K., et al., The intrinsic radioresistance of glioblastoma-derived cell lines is associated with a failure of p53 to induce p21(BAX) expression. Proc Natl Acad Sci U S A, 1998. 95(24): p. 14453-8.
9. Laitio, R., et al., Effect of Inhaled Xenon on Cerebral White Matter Damage in Comatose Survivors of Out-of-Hospital Cardiac Arrest: A Randomized Clinical Trial. JAMA, 2016. 315(11): p. 1120-8.
10. Arola, O. J., et al., Feasibility and cardiac safety of inhaled xenon in combination with therapeutic hypothermia following out-of-hospital cardiac arrest. Crit Care Med, 2013. 41(9): p. 2116-24.
11. Azzopardi, D., et al., Moderate hypothermia within 6 h of birth plus inhaled xenon versus moderate hypothermia alone after birth asphyxia (TOBY-Xe): a proof-of-concept, open-label, randomised controlled trial. Lancet Neurol, 2015.
12. Britton, G. L., et al., In vivo therapeutic gas delivery for neuroprotection with echogenic liposomes. Circulation, 2010. 122(16): p. 1578-87.
13. Peng, T., et al., Therapeutic time window and dose dependence of xenon delivered via echogenic liposomes for neuroprotection in stroke. CNS Neurosci Ther, 2013. 19(10): p. 773-84.
14. Maze, M., Preclinical neuroprotective actions of xenon and possible implications for human therapeutics: a narrative review. Can J Anaesth, 2016. 63(2): p. 212-26.
15. Sanders, R. D. and M. Maze, Xenon: from stranger to guardian. Curr Opin Anaesthesiol, 2005. 18(4): p. 405-11.
16. Esencan, E., et al., XENON in medical area: emphasis on neuroprotection in hypoxia and anesthesia. Med Gas Res, 2013. 3(1): p. 4.
17. Uberti, D., et al., p53 is dispensable for apoptosis but controls neurogenesis of mouse dentate gyrus cells following gamma-irradiation. Brain Res Mol Brain Res, 2001. 93(1): p. 81-9.

1. A method of protecting non-neoplastic cells from cellular damaging effects of a brain cancer directed therapy, said method comprising the steps of: a) obtaining a Noble Gas composition with neuro protective and/or regenerative potential; and b) administering said composition in a manner to promote regeneration and/to provide selective protection of non-malignant tissue from effects of chemotherapy and/or radiation therapy.

2. The method of claim 1, wherein said brain cancer directed therapy comprises therapies selected from a group comprising of: a) radiation therapy; b) chemotherapy; c) surgery; d) metabolic therapy; e) targeted therapy, and f) immunotherapy.

3. The method of claim 1, wherein said Noble Gas composition is gas mixture containing oxygen and a proportion by volume of 20 to 70% of xenon.

4. The method of claim 3, wherein said proportion of xenon is between 22 and 60% by volume to oxygen.

5. The method of claim 4, wherein said proportion of xenon is between 25 and 60% by volume to oxygen.

6. The method of claim 1, wherein said noble gas containing mixture consists only of a) oxygen and xenon or b) air and xenon.

7. The method of claim 1, wherein said noble gas containing mixture also contains nitrogen, helium, Nitric Oxide, krypton, argon or neon.

8. The method of claim 1, wherein said noble gas containing mixture contains a proportion by volume of oxygen of between 15 and 25%.

9. The method of claim 1, wherein said noble gas containing mixture is supplied for inhalation from a pressurized container at a pressure greater than 2 bar.

10. The method of claim 1, wherein said noble gas containing mixture is administered intranasally.

11. The method of claim 1, wherein said noble gas containing mixture is administered through the use of a hyperbaric chamber.

12. The method of claim 11, wherein said hyperbaric chamber is pressurized to a pressure of no more than 3 atm (0.3 MPa).

13. The method of claim 12, wherein a noble gas is administered to the patient while the patient is in the hyperbaric environment.

14. The method of claim 1 wherein said noble gas is administered by inhalation or simulated inhalation.

15. The method of claim 1, wherein said noble gas is xenon, helium, or a mixture of xenon and helium.

16. The method of claim 1, wherein the noble gas is xenon or a mixture of xenon and helium, and the partial pressure of xenon is no more than about 0.8 atm (0.08 MPa).

17. The method of claim 1, wherein said noble gas is administered mixed with air, the air partial pressure being about 1 atm (0.1 MPa).

18. The method of claim 1, wherein said noble gas is administered as part of a gas mixture comprising oxygen, the nitrogen partial pressure in the mixture being equal to or less than about 0.8 atm (0.08 MPa).

19. The method of claim 18, wherein said gas mixture is essentially free of nitrogen.

20. The method of claim 19, wherein the oxygen partial pressure is about 0.2 atm (0.02 MPa).
